(19) United States
(12) Patent Application Publication
(10) Pub. No.: US 2021/0186372 A1
(43) Pub. Date: Jun. 24, 2021

(54) A Method and Apparatus for Interpreting Multi-Breath Nitrogen Washout Data

(71) Applicant: University of Vermont and State Agricultural College, Burlington, VT (US)

(72) Inventor: Jason T BaTES, South Burlington, VT (US)

(21) Appl. No.: 16/757,481

(22) PCT Filed: Oct. 25, 2018
(86) PCT No.: PCT/US18/57438

§ 371 Date: Apr. 20, 2020

(51) Int. Cl. A61B 5/091; A61B 5/087; A61B 5/097

(52) U.S. Cl. CPC A61B 5/091; A61B 5/097; A61B 5/087

(57) ABSTRACT

A novel method and apparatus for analyzing multi-breath nitrogen washout (MBNW) data from a lung is provided. The novel method includes fitting multi-compartment lung model, having five free parameters, to an exhaled nitrogen concentration profile over the entire duration of expiration for each breath from the lung. The five free parameters include 1) functional residual capacity, 2) dead space volume, 3) the standard deviation of the rate of change of fractional contribution to expired flow from each lung region as a function of lung volume, 4) the intrinsic slope of Phase-Ill due to acinar asymmetry, and 5) the coefficient of variation of regional specific ventilation.

### CROSS-REFERENCE TO RELATED APPLICATIONS

The present application is related to, claims the earliest, available effective filing date(s) from (e.g., claims earliest available priority dates for other than provisional, patent applications; claims benefits under 35 USC § 119(e) for provisional patent applications), and incorporates by reference in its entirety all subject matter of the following listed application(s) (the “Related Applications”) to the extent such subject matter is not inconsistent herewith; the present application also claims the earliest available effective filing date(s) from, and also incorporates by reference in its entirety all subject matter of any and all parent, grandparent, great-grandparent, etc. applications of the Related Application(s) to the extent such subject matter is not inconsistent herewith:

U.S. provisional patent application 62/576,825, entitled “A Method for Interpreting Multi-Breath Nitrogen Washout Data”, naming Jason H. T. Bates as inventor, filed 25 Oct. 2017.

### STATEMENT REGARDING GOVERNMENT LICENSE RIGHTS

“This invention was made with government support under Ro1. HL1.30847 awarded by NIH-NHLBI. The U.S. government has certain rights in the invention.”

### BACKGROUND

### 1. Field of Use

The This invention generally relates to a method and system for non-invasively measuring pulmonary function, and more particularly to a method and system for analysis based on a multi-compartment model of the lung that accounts for the entire exhaled nitrogen profile, including Phases I (dead space washout), II (transition) and III (alveolar gas).

### 2. Description of Prior Art (Background)

Nitrogen washout of the lungs, produced by breathing pure oxygen, has been employed for decades in various forms as a means of assessing the nature pulmonary ventilation. In its simplest manifestation it can be used to determine Functional Residual Capacity (FRC) from the gas dilution inherent in the sequential decay of alveolar nitrogen plateaus from breath to breath, when breathing is perfectly regular and the lungs are uniformly ventilated, this decay is exponential with a rate constant directly relatable to FRC. If multiple compartments are involved that wash out at different rates then the decay is multi-exponential.

Information about regional ventilation in the lung is also provided by the shape of a single alveolar nitrogen plateau, although here the situation is more complicated. The slope of the nitrogen Phase-III is always positive for reasons that intrigued researchers for many years. The most obvious potential explanation for this slope is variations in the contributions of different lung regions to flow at different points in time, with those relatively under-ventilated regions (i.e., with higher nitrogen fraction (FN2)) contributing relatively more later in expiration. Much of the differences in regional ventilation were originally ascribed to gravity, the upper lung regions supporting the lower regions and therefore being more distended at FRC and thus with less room to further expand during inspiration.

Studies of nitrogen washout in microgravity, however, showed that although the slope of Phase-III is markedly diminished in weightlessness, a significant positive slope remains, so gravity is not the sole culprit. Indeed, this was expected on the basis of classic earlier studies which showed that interactions between diffusive and convective gas transport in the lung periphery gives rise to a positive Phase-III slope purely on the basis of asymmetries in parallel acinar structures.

Interest in the use of nitrogen washout to study ventilation heterogeneity in the lung has been rekindled in recent years by combining the information inherent in single-breath and multi-breath nitrogen washout maneuvers and analyzing not only the slope of Phase-III, but also how this slope changes from breath to breath as nitrogen is washed from the lungs. This analysis provides the parameters S_{cond} and S_{acin}, the former reflecting the rate at which regional differences in alveolar nitrogen develop due to time-constant differences of parallel lung regions fed by the conducting airways, and the latter reflecting structural asymmetry at the level of the acinus.

The relationship between the volume-normalized Phase-III slope of exhaled nitrogen fraction (FN2) versus lung turnover (the cumulative lung volume since the beginning of the test) allows the determination of the two parameters of physiological significance. One of these parameters, S_{acin}, is the intercept of a line fitted to the slope turnover relationship, while the other, S_{cond}, is the slope of the relationship. S_{acin} reflects structural asymmetry in the very distal airways of the lung, while S_{cond} reflects the degree of ventilation heterogeneity in the lung arising from time-constant differences caused by regional differences in conducting airway resistance.

Nevertheless, the clinical usefulness of the MBNW procedure is limited by the challenges associated with identifying Phase-III in each breath of a Multi-Breath Nitrogen Washout (MBNW) maneuver. There is no sharp demarcation between when Phase-II ends and Phase-HI begins because, for example, different lung regions have different volumes of anatomic dead space that start contributing alveolar gas to the expirate at different points in expiration. Typically, this means that the expired volumes in a MBNW maneuver must be larger than in normal resting breathing to ensure that the gas observed at the end of expiration is essentially all alveolar in origin. This can make the MBNW maneuver challenging for some subjects to perform. Even with large expiratory volumes, however, it is arbitrary as to when one decides that Phase-III has truly begun, particularly in pathological situations in which variations in regional emptying can be large.

The MBNW test, as it is currently practiced, requires that the subject breath in a regular manner such that the volume of oxygen inhaled each breath is as close to constant as possible. It also requires that the inhaled volume be significantly greater than the dead space volume so that Phase-Ill can be readily identified. These requirements place a burden on the test subject that, while not particularly difficult for normal adults to satisfy, may prove troublesome for young children and those with lung disease. Furthermore, the determination of the slope of Phase-HI requires that a decision be made as to when Phase-II ends and Phase-III begins, something that is arbitrary and may be problematic in heterogeneous lungs when the progression from Phase-II to Phase-III is gradual.

Thus, there is a need to overcome the methodological limitations of the traditional MBNW test and analysis, and account for the entire exhaled nitrogen profile, including Phases I, II and III.

### BRIEF SUMMARY

The invention is a novel method for analyzing multi-breath nitrogen washout data from the lung. Current methods of analysis are entirely empirical and are based on estimating the slope of Phase-Ill of the washout (Phase-III is the portion of the nitrogen concentration curve measured at the mouth over the final stages of each expiration). The limitations of the current approach are that it 1) requires subjects to breathe deeply and regularly, and 2) requires a subjective decision as to when the Phase-II portion of expiration (when dead space gas is being, exhaled) ends and the Phase-III portion begins. The invention disclosed herein overcomes both these limitations by fitting a multi-compartment model to the exhaled nitrogen concentration profile over the entire duration of expiration for each breath. The model can be fit successfully to measurements of nitrogen concentration at the mouth and changes in lung volume throughout a multi-breath nitrogen washout maneuver because it has only 5 free parameters: 1) functional residual capacity, 2) dead space volume, 3) the standard deviation of the rate of change of fractional contribution to expired flow from each lung region as a function of lung volume. 4) the intrinsic slope of Phase-III due to acinar asymmetry, and 5) the coefficient of variation of regional specific ventilation. The method thus provides several parameters of physiological importance while being applicable to data from subjects who are not breathing regularly and for whom the point of transition between Phase-II and Phase-III is not clear (which is often the case in lung disease).

The invention is also directed towards an apparatus for measuring and interpreting a patient's multi-breath nitrogen washout (MBNW) data. The apparatus includes a non-rebreathing valve; a T-nozzle having two selectable inlet ports and an outlet port, wherein the outlet port is connected to the non-rebreathing valve, and wherein one inlet port is connectable to a pure Oxygen source and wherein the other inlet port is connectable to ambient air source. The apparatus also includes a flowmeter connected to the non-rebreathing valve; and a microprocessor connected to the flowmeter, and wherein the microprocessor is connected to the non-rebreathing valve via a gas sampling line. The microprocessor includes instructions for determining five free parameters: V(0), V_{D}, σ_{b}, A and μ. V(0) represents the FRC of the subject, V_{D} represents the volume of the physiologic dead space, σ_{b} reflects the heterogeneity of lung emptying as a function of lung volume, A reflects the heterogeneity of regional tidal volume throughout the lungs, and μ is a reflection of structural asymmetry at the level of the acinus. The microprocessor also includes instructions for applying the five free parameters to the patient's MNBW data to determine functional lung capacities and ventilation heterogeneities.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken, in conjunction with the accompanying drawings in which:

FIG. 1 is a schematic of the key elements of the method for simulating MBNW data in accordance with the invention described herein;

FIG. 2 is a graphic example of expired F(t) predicted by the method in FIG. 1;

FIG. 3A is a graphic example of the first three breaths of F(t) during a MBNW maneuver predicted by the method in FIG. 1;

FIG. 3B is slope graph of the lines fitted to each expiratory F(t) versus the mean value of F(t) from the data shown in FIG. 3A;

FIG. 4 is FN2 data from a human subject showing the portions of the alveolar plateaus used for analysis after expiration of the first 300 ml of each breath;

FIG. 5A is FN2 data from a human subject showing the portions of the alveolar plateaus used for analysis after expiration of the first 200 ml of each breath;

FIG. 5B is FN2 data from a human subject showing the portions of the alveolar plateaus used for analysis after expiration of the first 100 ml of each breath;

FIG. 6 is an example model fit to MBNW data from a human subject;

FIG. 7 is a second example model fit to MBNW data from a human subject; and

FIG. 8 (prior art) is a schematic diagram illustrating a conventional setup for multiple-breath inert gas wash-ill/wash-out tests for determination of FRC and ventilation distribution (LCI) as known in the art

### DETAILED DESCRIPTION

The following brief definition of terms shall apply throughout the application:

The term “comprising” means including but not limited to, and should be interpreted in the manner it is typically used in the patent context;

The phrases “in one embodiment,” “according to one embodiment,” and the like generally mean that the particular feature, structure, or characteristic following the phrase may be included in at least one embodiment of the present invention, and may be included in more than one embodiment of the present invention (importantly, such phrases do not necessarily refer to the same embodiment);

If the specification describes something as “exemplary” or an “example,” it should be understood that refers to a non-exclusive example; and

If the specification states a component or feature “may,” “can,” “could,” “should,” “preferably,” “possibly,” “typically,” “optionally,” “for example,” or “might” (or other such language) be included or have a characteristic, that particular component or feature is not required to be included or to have the characteristic.

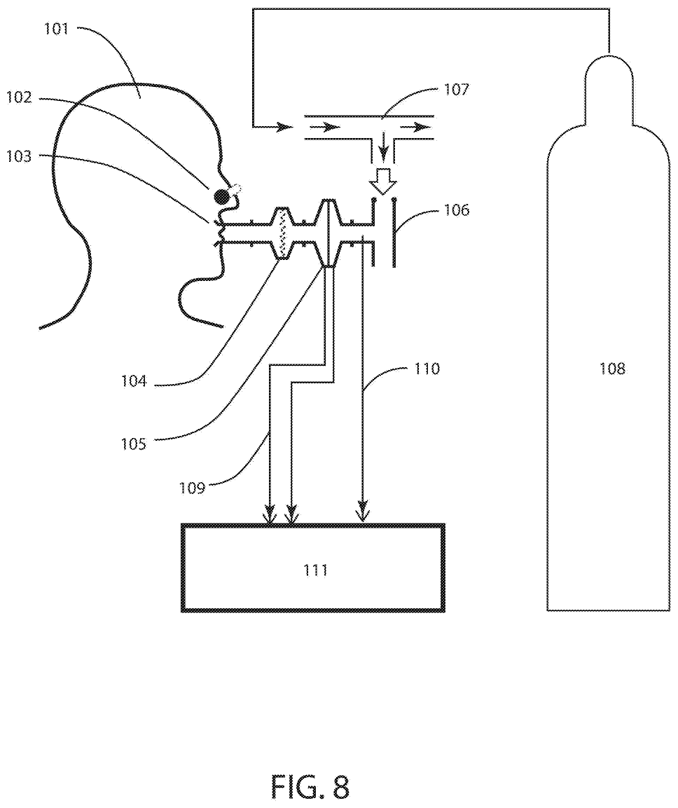

FIG. 8 is a schematic diagram illustrating a setup for multiple-breath inert gas wash-in/wash-out tests for measuring and interpreting a patient's multi-breath nitrogen washout (MBNW) data. A patient 101 having the nose occluded with a nose clip 102 breathes through a mouthpiece 103, a bacterial filter 104, a respiratory flowmeter 105 and a non rebreathing valve assembly 106. The Oxygen reservoir 108 is coupled to assembly T-nozzle 107 via a gas line. Flowmeter connection(s) 109 and a gas sample line 110 are also part of the setup.

To perform a multiple-breath inert gas wash-in/wash-out test, the test subject or patient 101 inspires ambient air from through assembly 107 (Oxygen connection is closed) through the non-rebreathing valve or one-way assembly 106. The non-rebreathing valve assembly 106 is constructed by one-way valves allowing gas to flow in one direction only. Because of the construction of the valve 106, the test subject does not exhale gas back to assembly 107 during exhalation. The test subject 101 may use a face mask instead of nose clip 102 and mouthpiece 103. The microprocessor unit 111 consists of a measuring apparatus comprising flowmeter electronics; and, at least one gas analyzer and coded instructions for analyzing multi-breath nitrogen washout data real-time by fitting a multi-compartment model to the exhaled nitrogen concentration profile over the entire duration of expiration for each breath.

A test consists of a period where the test subject inspires ambient air through assembly 107 and exhales to the surrounding air a number of times (wash-in period) followed by a period where the test subject is breathing Oxygen (wash-out period). During the testing (both during the wash-in and the wash-out period) the concentration in the inhaled and/or exhaled air of the inert gas in the mixture is measured by a fast responding gas analyzer. Instead of gas concentration the gas analyzer may equally well measure the partial pressure of the gas. The partial pressure can be obtained from the fractional concentration of dry gas or any other measure of gas concentration or pressure using appropriate conversion factors as known in the art. Also, the flow of the inhaled and/or exhaled air is measured by means of the flowmeter 105. These measurements are made continuously real-time. Fitting the multi-compartment model invention to the exhaled nitrogen concentration profile over the entire duration of expiration for each breath is described herein.

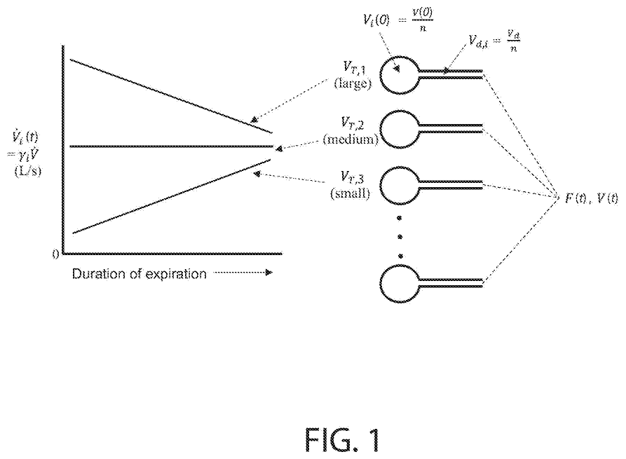

Referring also to FIG. 1 of the drawings, a human lung is modeled as a collection of n parallel alveolar units served by individual airways that intersect at the airway opening. A parallel collection of units with identical functional residual capacities, Vᵢ(0), and individual dead space volumes, V_{d,i}, connect at the airway opening where a flow-weighted sum of the contributions from each of the units add to produce the mole fraction of nitrogen, F(t), measured at the mouth as lung volume, V(t), cycles through the MBNW maneuver.

The fraction contribution to expired flow from each unit, γ1, determines the individual unit flows, V′ᵢ(t), according to the individual unit tidal volumes, V_{T,i}, such that units with high tidal volumes contribute fractional flows that decrease linearly as V(t) decreases throughout expiration, while units with low tidal volumes contribute fractional flows that increase linearly. This ensures the slope of Phase-III is positive and that this slope increases as regional differences in V′ᵢ(t) increase.

The volume of an airway remains constant and thus constitutes the fixed anatomic dead space of the unit it serves. The unit dead space volumes are all identical and thus each equal to V_{D}/n, where V_{D} is the total anatomic dead space of the model.

The functional residual capacities of the units are also identical and thus are determined by the total functional residual capacity (FRC) of the model divided by n. The method performs a MBNW maneuver by having the total volume, V(t), cycle over a number of consecutive breaths. Pure oxygen enters the airway opening during each inspiration of the maneuver, while the mole fraction of nitrogen, F(t), leaving the airway opening is calculated real time by microprocessor (FIG. 8-111) during each expiration. The mole fractions, Fi(0) (i=1, 2, . . . , n) of nitrogen in each unit at the start of the MBNW maneuver (i.e, at t=0 when the first inspiration of O₂ begins) are identical and equal to the ambient value of 0.79.

The MBNW maneuver is performed with regular breathing; V(0) is equal to FRC and the excursions in V(t) during each breath in the maneuver (i.e., tidal volume, V_{T}) are identical. In this case, the initial volumes of each unit are identical and equal to V(0)/n, and the only functional attribute that distinguishes one unit from another is, its individual tidal volume, V_{T,i}(see FIG. 1).

The slope of Phase III is always positive, so the relative contributions of units with high specific ventilation (i.e., those with large V_{T,i}) must increase progressively as expiration proceeds compared to units with low specific ventilation (i.e., those with small V_{T,i}).

Accordingly, we let the units with lower than average V_{T,i} make fractional contributions, γᵢ(t), to the total flow, V(t), that increase linearly with the decrease in V(t) during expiration. The rate of increase of γᵢ(t) is inversely proportional to V_{T,i}. The converse applies for units with greater than average V_{T,i}. Consequently, if a unit has a γᵢ(t) that decreases throughout expiration, its tidal volume, and thus its mean value of γᵢ(t), is higher than that of a unit whose γᵢ(t) increases throughout expiration, as illustrated in FIG. 1. It is convenient to express γᵢ(t) relative to the value of V(t) at the midpoint of its range throughout expiration. The midpoint volume, V̅, is

V̅=V(0)+V_{T}/2 (1)

which gives

γᵢ(t)=aᵢ+bᵢ[(V(t)−V̅)/V̅] (2)

where aᵢ and bᵢ are dimensionless constants. This causes γᵢ(t) to be antisymmetric relative to V(t) σbout V̅, resulting in the contribution to V_{T,i} from the term in bᵢ in Eq. 2 averaging to zero over expiration. Consequently, aᵢ alone is equal to V_{T,i} as a fraction of V_{T}.

The bᵢ are chosen from a zero-mean Gaussian distribution with standard deviation σb. The aᵢ vary about their mean value of 1/n by an amount proportional to their respective bᵢ, with constant of proportionality A. That, is,

aᵢ(1/n)=Abᵢ (3)

which gives

γᵢ(t)=(1/n)+bᵢ[A+(V(t)/V̅)−1] (4)

Equation 4, however, makes it possible for γᵢ to achieve physically meaningless negative values in those very high ventilation units whose contributions decrease sufficiently rapidly throughout expiration. To avoid this, we impose the condition that if γᵢ(t) ever reaches zero, it remains there until V(t) returns to the point where γᵢ(t) becomes positive again. Nevertheless, the fractional contributions to the total flow from all the units must always sum to provide the total flow. Accordingly, whenever γᵢ(t) becomes zero for some of the units, the remaining γᵢ(t) are scaled to maintain their summed contributions at unity. The definition of γᵢ(t) thus becomes

$$\begin{matrix}{γ}_{i} ( t ) = α ( t ) \{ ( 1 / n ) + {b}_{i} [ A + ( V ( t ) / \overline{V} ) - 1 ] \} & ; A + ( V ( t ) / \overline{V} ) > 1 , \\ = 0 & ; A + V ( ( t ) / \overline{V} ) \leq 1.\end{matrix} \qquad ( 5 )$$

The function α(t) is chosen at so that, at each value of t,

Σᵢ₌₀ⁿ(γᵢ(t)=1) (6)

Therefore, α(t)=1 whenever none of the γᵢ(t) are zero, but α(t)>1 otherwise.

The model is driven by a specified V(t) signal, from which V′(t) is determined by numerical differentiation. The flow, V′ᵢ(t), into each unit is then determined as

V̅i′(t)=γᵢ(t)V′(t) (7)

using γᵢ(t) from Eq. 5, and is numerically integrated to give the unit volume, Vᵢ(t). The mole fraction of nitrogen in each unit, Fᵢ(t), is the ratio of the volume of nitrogen in the unit divided by Vᵢ(t). Inspiration begins with the volume of nitrogen in each unit increasing due to the inhalation of the gas in its airway dead space which has the same nitrogen fraction as the unit itself had during the previous expiration. Once the dead space gas has passed back into a unit, however, the volume of nitrogen it contains stays constant for the remainder of inspiration because only pure oxygen is inhaled thereafter. Fᵢ(t) thus decreases as the fixed volume of nitrogen becomes progressively diluted by oxygen. During expiration, Fᵢ(t) remains constant but the volume of nitrogen in each unit decreases at a rate given by the product of Vᵢ(t) and Fᵢ(t). These various situations are expressed mathematically as

$$\begin{matrix}d [ {V}_{i} ( t ) {F}_{i} ( t ) ] / ( dt ) = {V}_{i}^{′} ( t ) {F}_{i} ( t ) & ; {V}_{i}^{′} \geq 0 and inhaled volume \leq {V}_{Di} , \\ = 0 & ; {V}_{i}^{′} \geq 0 and inhaled volume > {V}_{Di} , \\ = {V}_{i}^{′} ( t ) {F}_{i} ( t ) & ; {V}_{i}^{′} < 0.\end{matrix} \qquad ( 8 )$$

During inspiration, the nitrogen mole fraction, F(t), at the common entrance to the unit airways (i.e., the equivalent of the mouth) is zero. During expiration, F(t) is a flow-weighted sum of the nitrogen mole fractions, F_{d,i}(t), in each unit dead space (i.e. the individual unit airways). Early in expiration, F_{d,i}(t)=0 because each dead space is filled with pure oxygen from the previous inspiration, but once a unit empties itself of oxygen the dead space becomes filled with gas from the unit in which case F_{d,i}(t)=Fᵢ(t). That is,

F(t)=0; V′(t)≥0

F(t)=Σ₁ⁿ(γᵢ(t)V′(t)F_{d,i}(t)); V′(t)<0. (9)

So far we have been assuming that Fᵢ(t) does not vary with time during expiration. This is not strictly true for several reasons, but by far the most important reason for the purposes of simulating F(t) is the diffusive-convective interaction within the structurally asymmetric acinus, that has been described as a form of “diffusive pendelluft” (Engel, J A P 1983), and which is responsible for the finite value of the parameter Sac/n determined conventionally. We represent this phenomenon by; replacing the constant value of Fᵢ(t) throughout expiration with a quantity \hat{F}ᵢ(V) that increases linearly as V(t) decreases throughout expiration, and which is symmetric about V̅. That is,

\hat{F}ᵢ(V)=μFᵢ[1−V(t)/V̅]+Fᵢ. (10)

The constant of proportionality, μ, is assumed to be the same for all units.

Strictly speaking, \hat{F}₁(t) should replace Fᵢ(t) in the last line of Eq. 8. \hat{F}₁(t) and become F_{d,i}(t) in Eq. 9, but this would involve the significant computational complexity of determining how the nitrogen mole fraction in each unit, dead space changes throughout expiration due to a mole fraction input from its unit that varies with time. We elect, not to do this, in the interests of simplicity, on the grounds that the symmetry of Eq. 10 about V̅ means that the volume of nitrogen exhaled from a unit into its dead space during an entire expiration is the same as if \hat{F}₁(t)=Fᵢ.

A prime motivation for creating this model is to deal with the fact that subjects performing MBNW maneuvers inevitably exhibit breath-to-breath variabilities in end-expiratory lung volume and tidal volume. To make the model applicable to this general situation, we replace V̅ and V_{T} as defined in Eq. 1 with the midpoint of V(t) and the mean tidal volume, respectively, during a MBNW maneuver.

The above model has only five free parameters—V(0), V_{D}, σ_{b}, A and μ). V(0) represents the FRC of the subject, V_{D} represents the volume of the physiologic dead space, σ_{b} reflects the heterogeneity of lung emptying as a function of lung volume, A reflects the heterogeneity of regional tidal volume throughout the lungs, and μ is a reflection of structural asymmetry at the level of the acinus. The parameters σ_{b} and A can further be combined into a measure of the coefficient of variation of regional specific ventilation throughout the lungs C_{V·E}, as follows. First note that the specific ventilation, V_{E′,i}, of a unit is the ratio of its tidal volume to its functional residual capacity, which is (using Eq. 3)

$$\begin{matrix}{V}_{{E}^{′} , i} = {V}_{T , i} / ( V ( 0 ) / n ) \\ = {na}_{i} {V}_{T} / V ( 0 ) \\ = ( n ( A {b}_{i} + ( 1 / n ) ) {V}_{T} ) / V ( 0 ) .\end{matrix} \qquad ( 11 )$$

C_{V′}_{_{E}} is the standard deviation of V_{E′,i} normalized to its mean, the latter being, simply the specific ventilation of the entire lung, namely V_{T}/V(0). This gives, from Eq. 11,

C_{V′}_{_{E}}=nAσb (12)

### Model Fitting

Because the model has 5 free parameters it is practical to consider fitting it to measurements of V′(t) and F(t) from subjects performing MBNW maneuvers, with the initial conditions being the common mole fraction of nitrogen in each unit at t=0. The model is fitted using a sequential grid-search procedure in which the root mean squared residual, R, between the measured and model-predicted F(t) is minimized over a grid of V(0) and V_{D} values encompassing their likely ranges while σ_{b}, A and μ are set equal to zero. With the values of V′(t) and F(t) set at their best-fit values, a second search is performed over a grid of σ_{b} and A values, followed by a search over possible values for μ. The entire procedure is then repeated on finer grids until R ceases to change by more than the forth significant digit, at which point R is considered to have achieved its minimum value of Rₘᵢₙ.

The sensitivity of the fit to each parameter is determined by varying each parameter in turn by ±5% either side of its best-fit value and determining the mean of the two resultant changes in RMSR, denoted ΔR. The strength, Sₚ by which the data determined the value of parameter p is expressed as the ratio of the fractional change in R to the fractional change in p. That is,

Sp=ΔR/(0.05Rₘᵢₙ) (13)

where p is any one of V(0), V_{D}, σ_{b}, A or μ. We found, however, that the two parameters cm, A tend to compensate for each other, which can be understood by the appearance of their product in Eq. 5. In other words, the two parameters can often vary in opposite directions by substantial amounts without dramatically affecting the quality of the fit. Furthermore, while these two parameters are based on the idealization of the lung as a parallel set of compartments differing only in their tidal volumes and fractional contributions to expired flow, they are nevertheless not directly interpretable in terms of recognized physiological quantities that, are readily verifiable by other means. Their product, on the other hand, is not only more robust but also gives rise to an estimate of regional heterogeneity of specific ventilation, C_{V′}_{_{E}} (Eq. 12) that has a physiological interpretation of direct relevance to lung pathology. Accordingly, the primary outputs of model fitting are the parameters V(0), V_{D}, μ, and C_{V′}_{_{E}}.

### Results

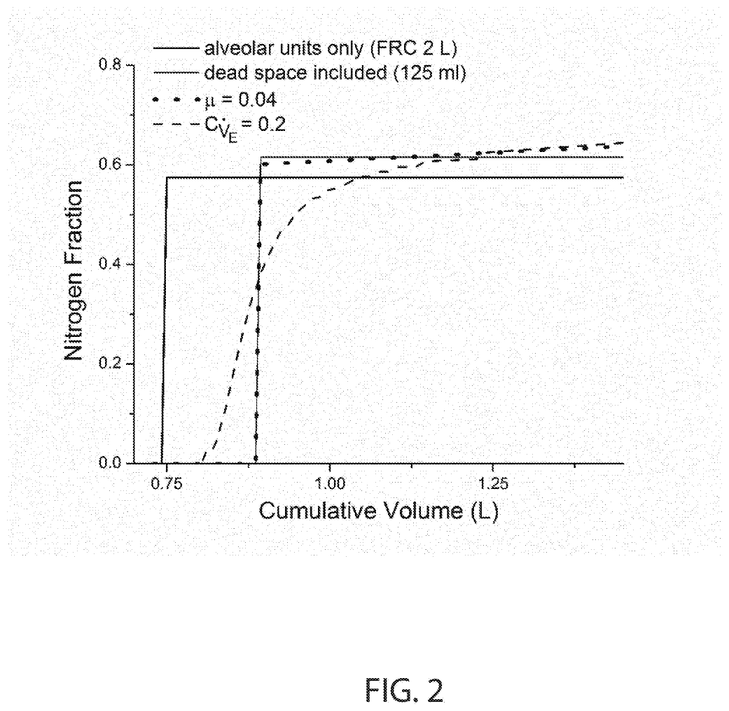

FIG. 2 illustrates the model predictions of F(t) during, the first expiration of a simulated MBNW maneuver, with V_{T}=0.75 L and V(0)=2 L, the Phase-III plateau in F(t) is horizontal throughout expiration at a level determined by the dilution of 2 L of resident alveolar gas by 0.75 l of pure oxygen (thick solid line in FIG. 2). When a finite dead space volume is introduced (V_{D}=0.15 L) Phase-III is still horizontal but at an elevated level because now only 0.60 L of oxygen dilutes the resident gas (thin solid line). With the introduction of the effects of acinar asymmetry (μ=0.04) Phase-III gains a positive slope (dotted line). Finally, regional heterogeneity in specific ventilation (σ_{b}=0.01, A=0.4) produces a sigmoidal shaped Phase-II that transitions smoothly into Phase-III (dashed line).

Stated differently, while still referring to FIG. 2, an example is shown of expired F(t) predicted by the model consisting only of identical alveolar units (thick solid line), with the addition of identical dead spaces to each unit (thin solid line), with the further addition of the effects of acinar asymmetry (i=0.04; dotted line), and with the further addition of the effects of regional heterogeneity in alveolar ventilation (σ_{b}=0.01, A=0.4; dashed line).

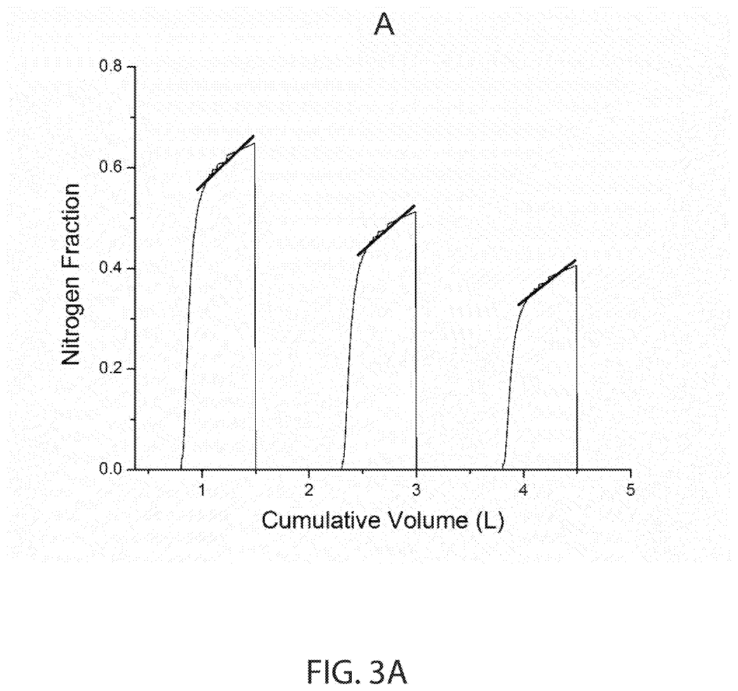

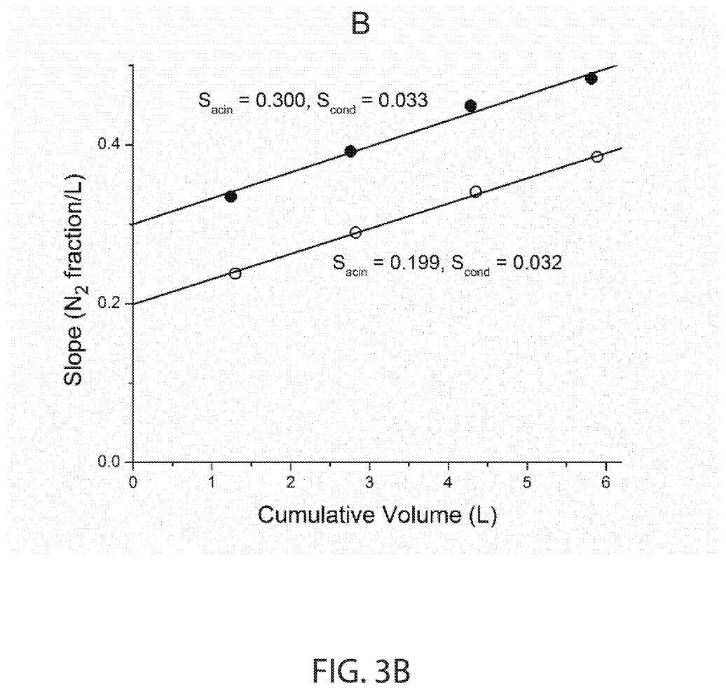

FIG. 3 illustrates the conventional MBNW analysis applied to model data. FIG. 3A shows the first three breaths of a maneuver with V(0)=2 L, V_{D}=0.15 L, σ_{b}=0.04, A=0.4 and μ=0.4. Also shown are straight line segments fit to Phase-III in each breath assuming that Phase-Ill begins after a volume equal to V_{D} has been exhaled and ends at the end of expiration. FIG. 3B shows the slopes of these line segments, normalized to their respective mean values of F(t) throughout Phase-III, versus mean cumulative volume. S_{cond} is the slope of this relationship, while S_{acin} is the intercept.

However, FIG. 3B also illustrates the importance of determining where Phase-Ill begins. It is clear from FIG. 3A that F(t) is not perfectly straight over the sections where the line segments have been fit, but rather has a downward concavity reflecting the gradual progression from Phase-II to Phase-III. If Phase-III is assumed to start later in expiration (at the point where 0.25 L of gas has been exhaled instead of 0.15 L) the net concavity is less, but the estimated value of S_{acin} is markedly reduced (FIG. 3B).

Stated differently, and still referring to FIG. 3A and FIG. 3b. FIG. 3A shows an example of the first three breaths of F(t) during, a MBNW maneuver predicted by the model (thin trace) with V(0)=2 L, V_{D} 0.15 L, σ_{b}=0.04, A=0.4 and μ=0.4. Also shown are the straight-line fits (thick lines) to each expiratory portion of F(t) from the point when a volume equal to V_{D} has been expired until the end of the expiration.

FIG. 3B shows the slopes of the lines fitted to each expiratory F(t) versus the mean value of F(t) from the data shown in FIG. 3A (closed circles) together with their linear fit. The slope of this relationship gives S_{cond} (0.033 L⁻¹) while its intercept with, the vertical axis is S_{acin} (0.30 L⁻¹). Also shown are the normalized slopes and linear fit obtained when the analysis illustrated in FIG. 3A is repeated assuming V_{D}=0.25 L. In this case, S_{cond}=0.032 L⁻¹ and S_{acin}=0.20 L⁻¹.

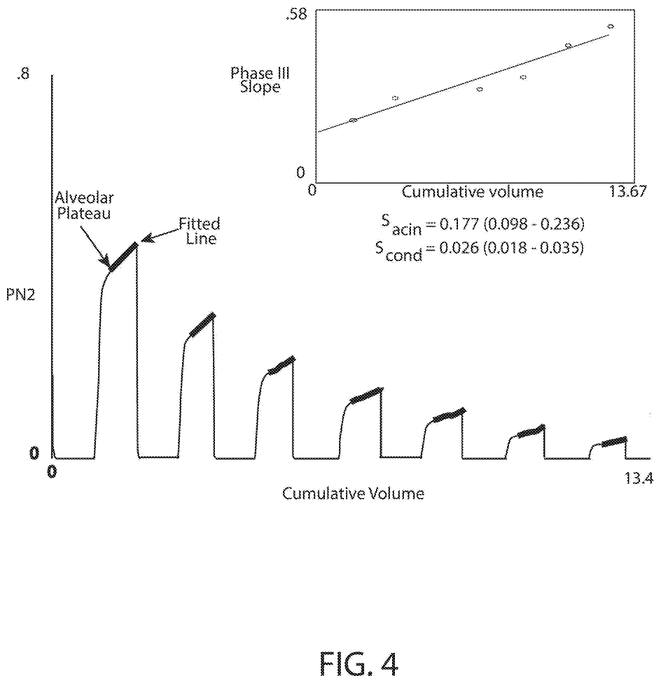

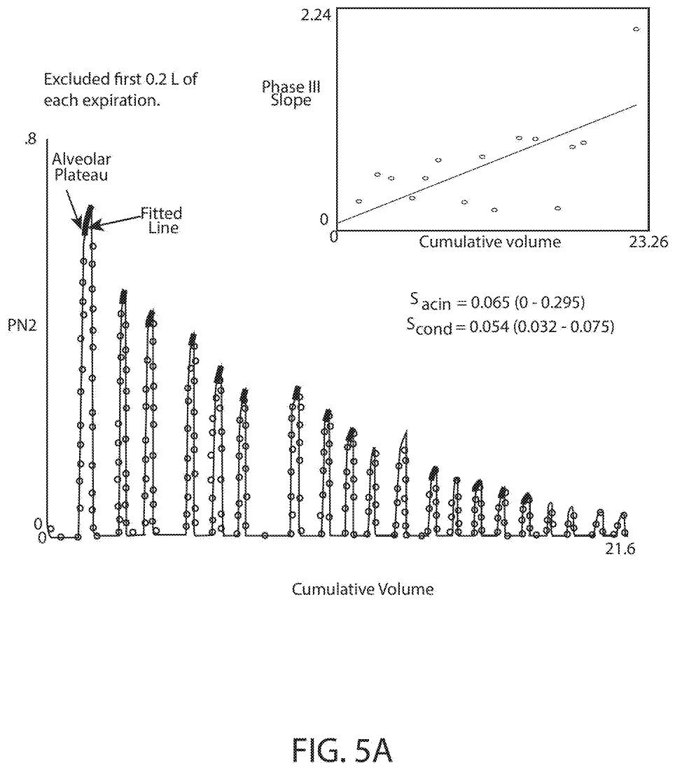

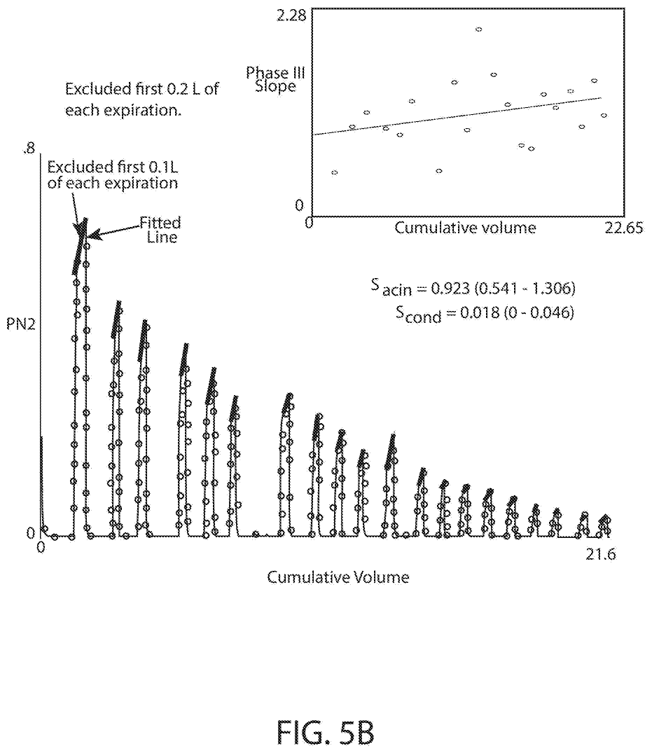

FIG. 4 shows the conventional analysis applied to data from a human subject. In this case, the alveolar plateaus in FN2 are quite well defined so calculating S_{cond} and S_{acin} is not problematic. In contrast, the data from another subject shown in FIG. 5 has poorly defined alveolar plateaus that give rise to poorly defined values for S_{cond} and S_{acin}. Furthermore, these values vary substantially with variations in the volume of the initial part of expiration that is discarded from analysis.

Stated differently and still referring to FIG. 4, FN2 data from a human subject (black). The portions of the alveolar plateaus, used for analysis (after expiration of the first 300 ml of each breath) and the fitted lines are indicated in FIG. 4. It will be appreciated that the portions of the alveolar plateaus used for analysis (after expiration of the first 300 ml of each breath) and the fitted lines are indicated in FIG. 4 are in close agreement. It will be further appreciated that all the alveolar plateaus shown in FIG. 4 are in close agreement with the fitted lines but only the first alveolar plateau and corresponding fitted line are indicated for clarity. The inset shows S_{cond} vs. S_{acin}.

Referring also to FIG. 5A and FIG. 5B, there is shown FN2 data from a human subject showing the portions of the alveolar plateaus used for analysis after expiration of the first 200 ml of each breath, and FN2 data from a human subject showing the portions of the alveolar plateaus used for analysis after expiration of the first 100 ml of each breath, respectively. It, will be further appreciated that all the alveolar plateaus shown in FIG. 5A and FIG. 5B are in close agreement with the fitted lines but only the first alveolar plateau and corresponding fitted line are indicated for clarity.

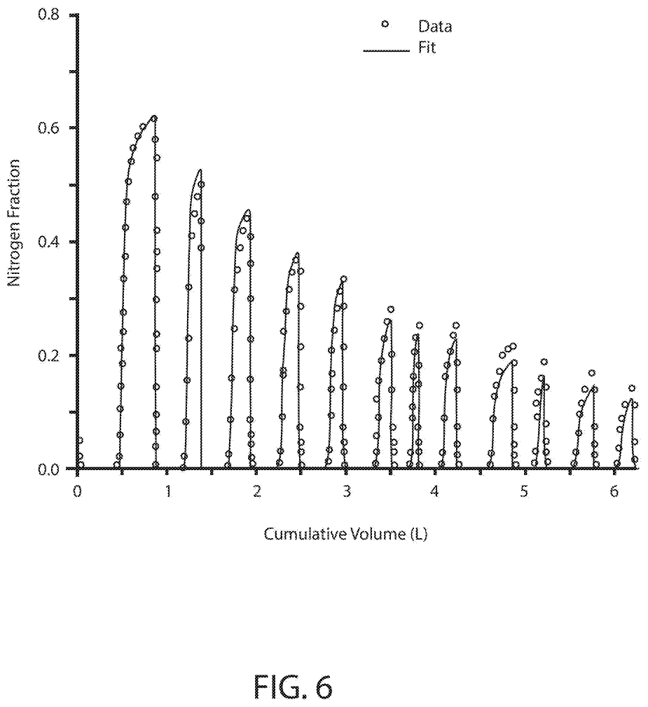

FIG. 6 shows an example model fit to MBNW data from a human subject. The breathing pattern is somewhat regular, although V(t) shows clear breath-to-breath variations in both tidal volume and end-expiratory volume. The best-fit model parameter values are V(0)=1.51 L, V_{D}=0.09 L, and μ=0.025, giving a value for C_{V′}_{_{E}} of 0.37. The RMSR between data and fit is 0.032.

The parameter sensitivities per Eq. 13 are S_{V(0)}=0.32, S_{V}_{_{d}}=0.26, S_{μ}=0.00, and S_{C}_{_{V′E}}=0.25. Thus, the value of μ is very weakly determined by these data; its value can vary widely with little effect on the quality of the fit. The other three parameters are somewhat more strongly determined by the data, although fractional variations in their values give rise to smaller fractional variations in RMSR.

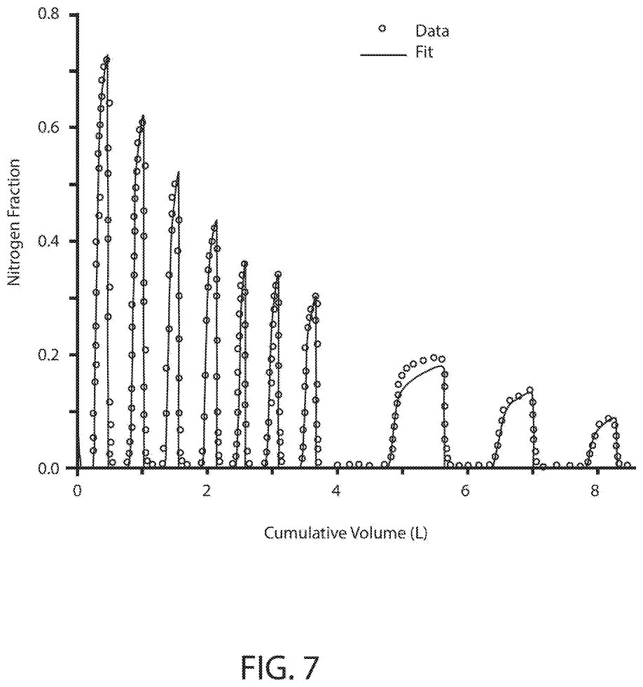

FIG. 7 shows another example fit to experimental data, this time with breathing that is much less regular. Standard analysis to derive meaningful values of Sacin, and Sacin would be impossible in this case, yet the model fit disclosed herein follows the vagaries of the data quite well and provides interpretable parameter values of V(0)=1.82 L, V_{D}=0.13 L, μ=0.069, A=1.97, and C_{V′}_{_{E}}=0.65. The RMSR between data and fit is 0.011. The parameter sensitivities per Eq. 13 are S_{V(0)}=1.16, S_{V}_{_{d}}=2.99, Sμ=0.00, and S_{C}_{_{V′E}}=1.21. Again, μ is very weakly determined by these data. In contrast, the other three parameters are quite strongly determined, since small fractional variations in their values produces greater fractional variations in RMSR.

### DISCUSSION

The present invention disclosed herein for analyzing MBNW data was motivated by the desire to avoid the practical issues previously mentioned. Accordingly, the invention discloses a microprocessor computational model of the lung of sufficient complexity to be able to simulate realistically appearing expiratory nitrogen profiles during a MBNW maneuver that is not limited by the need to identify the precise beginning of Phase-III but rather simulates the entirety of phases I, I and III.

It will be appreciated, that the model behavior must be governed by few enough free parameters that these parameters can be robustly estimated from a typical MBNW data set. The invention satisfies this requirement by developing a model having only five free parameters.

Nevertheless, the necessary simplicity of such a model represents numerous simplifying assumptions that collectively embody the main limitations of inventive approach. Perhaps most important of these is the representation, of the lung as a series of parallel compartments each with its own independent anatomic dead space. This approach simplifies the fact that the conducting airways are actually a tree structure, and that time constant differences between different parallel lung regions are not entirely independent of each other as the model assumes.

In addition, the invention assigns a common dead space volume to all these regions thus allowing the model to simulate a sigmoidal Phase-II without introducing additional free parameters. While is a gross oversimplification of reality this non-obvious inventive step produces realistic simulations. The invention also represents the complex phenomena involved in diffusive-convective gas transport interactions in the lung periphery as a fixed contribution to the Phase-III slope that is common to all regions of the lung regardless of their other differences.

It will be appreciated that the invention disclosed herein is a novel approach to the analysis of MBNW data from the lungs that overcomes at least two significant limitations of the current prior art approach, which are that 1) subjects must breathe deeply and evenly, and 2) a decision must be made as to when dead space gas has been fully expired during an exhalation and pure alveolar gas has started to appear at the mouth.

These limitations are significant because 1) subjects with significant lung disease and small children may not be able to breathe in a manner that is sufficiently deep and regular for the current prior art methods, and 2) there is no definitive point at which pure alveolar gas appears at the mouth during expiration, especially in diseased lungs, so the prior art methods have to make an empirical decision as to when this transition nominally occurs.

The invention disclosed herein avoids both these limitations by fitting a mechanistically based computational model of the lung to the entire expiratory nitrogen concentration from each breath in a multi-breath nitrogen washout maneuver. Furthermore, as noted earlier the prior art methods provide two parameters, known as Sa_{cin} and S_{cond}, that are presented as purely empirical reflections of regional heterogeneities in ventilation throughout the lung.

The invention disclosed herein, being based on a computational model of the lung, provides a measure of the degree of variation in regional specific ventilation throughout the lung, a quantity that has a clear physiological interpretation.

For example, C_{V′}_{_{E}} (Eq. 12) is related to S_{cond}, and provides a direct measure of regional ventilation heterogeneity.

It should be understood that the foregoing description is only illustrative of the invention. Thus, various alternatives and modifications can be devised by those skilled in the art without departing from the invention. Accordingly, the present invention is intended to embrace all such alternatives, modifications and variances that fall within the scope of the appended claims.

1. A method for interpreting a patient's multi-breath nitrogen washout (MBNW) data, the method comprising:
determining five free parameters, wherein the five free parameters comprise V(0), V_{D}, σ_{b}, A and μ wherein V(0) represents the FRC of the subject, V_{D} represents the volume of the physiologic dead space, σ_{b} reflects the heterogeneity of lung emptying as a function of lung volume, A reflects the heterogeneity of regional tidal volume throughout the lungs, and μ is a reflection of structural asymmetry at the level of the acinus; and
applying the five free parameters to the patient's MNBW data to determine functional lung capacities and ventilation heterogeneities.

2. The method as in claim 1 further comprising measuring a patient's V′(t)(measured) and F(t)(measured) parameters.

3. The method as in claim 2 further comprising predicting a patient's V′(t)(predicted) and F(t)(predicted) parameters.

4. The method as in claim 3 further comprising minimizing a root mean squared residual R between F(t)(measured) and F(t)(predicted).

5. The method as in claim 4 wherein minimizing R further comprises minimizing Rover a first grid comprising V(0) and V_{D} values while σ_{b}, A and μ are set equal to zero to determine R₁.

6. The method as in claim 5 wherein minimizing R further comprises minimizing R₁ over a second grid comprising σₕ and A values, followed by a search over possible values for μ.

7. An apparatus for measuring and interpreting a patient's multi-breath nitrogen washout (MBNW) data, apparatus comprising:
a non-rebreathing valve;
a T-nozzle having two selectable inlet ports and an outlet port, wherein the outlet port is connected to the non-rebreathing valve, and wherein one inlet port is connectable to a pure Oxygen source and wherein the other inlet port is connectable to ambient air source;
a flowmeter connected to the non-rebreathing valve; and
a microprocessor connected to the flowmeter, and wherein the microprocessor is connected to the non-rebreathing valve via a gas sampling line, wherein the microprocessor comprises instructions for:
determining five free parameters, wherein the five free parameters comprise V(0), V_{D}, σ_{b}, A and μ, wherein V(0) represents the FRC of the subject, V_{D} represents the volume of the physiologic dead space, σ_{b} reflects the heterogeneity of lung emptying as a function of lung volume, A reflects the heterogeneity of regional tidal volume throughout the lungs, and μ is a reflection of structural asymmetry at the level of the acinus; and
applying the five free parameters to the patient's MNBW data to determine functional lung capacities and ventilation heterogeneities.

8. The apparatus for measuring and interpreting a patient's multi-breath nitrogen washout (MBNW) data as in claim 7, wherein the microprocessor comprises further instructions for determining a patient's V′(t)(measured) and F (t) (measured) parameters via the flowmeter and the gas sampling line.

9. The apparatus as in claim 8, wherein the microprocessor comprises further instructions for predicting a patient's V′(t)(predicted) and F(t)(predicted) parameters.

10. The apparatus as in claim 9, wherein the microprocessor comprises further instructions for minimizing a root mean squared residual (RMSR) between F(t)(measured) and F(t)(predicted).

11. The apparatus as in claim 10, wherein minimizing RMSR between F(t)(measured) and F(t)(predicted) comprises further instructions for minimizing RMSR over a first grid comprising V(0) and V_{D} values while σ_{b}, A and μ are set equal to zero to determine RMSR₁.

12. The apparatus as in claim 11, wherein the microprocessor comprises further instructions for minimizing RMSR₁ over a second grid comprising σ_{b} and A values, followed by a search over possible values for μ.

13. An apparatus for fitting a multi-compartment model to a patient's multi-breath nitrogen washout (MBNW) data, the apparatus comprising:
a non-rebreathing valve;
a T-nozzle having two selectable inlet ports and an outlet port, wherein the outlet port is connected to the non-rebreathing valve, and wherein one inlet port is connectable to a pure Oxygen source and wherein the other inlet port is connectable to ambient air source;
a flowmeter connected to the non-rebreathing valve; and
a microprocessor connected to the flowmeter, and wherein the microprocessor is connected to the non-rebreathing valve via a gas sampling, line, wherein the microprocessor comprises instructions for:
modeling a human lung as a collection of n parallel alveolar units
determining from the n parallel alveolar units five free parameters, wherein the five free parameters comprise V(0), V_{D}, σ_{b}, A and μ, wherein V(0) represents the FRC of the subject, V_{D} represents the volume of the physiologic dead space, σ_{b} reflects the heterogeneity of lung emptying as a function of lung volume, A reflects the heterogeneity of regional tidal volume throughout the lungs, and μ is a reflection of structural asymmetry at the level of the acinus; and
applying the five free parameters to the patient's MNBW data to determine functional lung capacities and ventilation heterogeneities, wherein applying the five free parameters to the patient's MNBW data to determine functional lung capacities and ventilation heterogeneities further comprises:
further instructions for determining a patient's V′ (t)(measured) and F(t)(measured) parameters via the flowmeter and the gas sampling line; and
predicting a patient's V′(t)(predicted) and F(t)(predicted) parameters.

14. The apparatus as in claim 13, wherein the microprocessor comprises further instructions for minimizing a root mean squared residual (RMSR) between F(t)(measured) and F(t)(predicted) comprises minimizing RMSR over a first grid comprising V(0) and V_{D} values while σ_{b}, A and μ are set equal to zero to determine RMSR₁.

15. The apparatus as in claim 15, wherein the microprocessor comprises further instructions for minimizing RMSR₁ over a second grid comprising σ_{b} and A values, followed by a search over possible values for μ.
